(19) United States
(12) Patent Application Publication
(10) Pub. No.: US 2002/0090663 A1
(43) Pub. Date: Jul. 11, 2002

(54) Immunoassay for fish identification

(76) Inventors: James X. Hartmann, Boca Raton, FL (US); Fei Duan, New York, NY (US)

Correspondence Address: Stanley A. Kim, Akerman, Senterfitt & Eidson, P.A., P.O. Box 3188, 222 Lakeview Avenue, Suite 400, West Palm Beach, FL, 33402-3188, US

(21) Appl. No.: 09/990,718

(22) Filed: Nov. 21, 2001

Related U.S. Application Data
(60) Provisional application No. 60/252,444, filed on Nov. 21, 2000.

(51) Int. Cl. G01N 33/53; G01N 33/537; G01N 33/543

(52) U.S. Cl. 435/7.92; 436/525

(57) ABSTRACT

A lateral flow immunoassay device for identifying the presence of tissue from a particular species of billfish in a test sample includes a substrate onto which a billfish specific antigen-containing sample has been immobilized. The substrate has a first end having thereon the immobilized billfish specific antigen-containing sample and a second end adapted to receive a solution including an antibody that specifically binds the billfish specific antigen.

### CROSS REFERENCE TO RELATED APPLICATIONS

The present application claims the priority of U.S. provisional application No. 60/252,444 filed Nov. 21, 2000.

### FIELD OF THE INVENTION

The present invention relates to devices, methods, and kits for identifying a particular species of fish in a biological sample.

### BACKGROUND OF THE INVENTION

Billfishes, such as sailfish, marlin and spearfish, are among the largest marine fishes, and have both recreational and commercial value. Although their sale is illegal in the continental United States, billfishes have an annual worldwide commercial catch value of about one to two billion dollars and a consumer market value of about four to six billion dollars. There is a considerable black market in the transport and sale of the flesh from these fishes.

Atlantic billfish management strategies in the United States are guided by the International Commission for the Conservation of Atlantic Tunas (ICCAT), the National Marine Fisheries Service (NMFS), and the newly established Florida Fish and Wildlife Conservation Commission (FFWCC). ICCAT's Standing Committee on Research and Statistics (SCRS) reported in 1995 that the biomass for sailfish, blue marlin, and white marlin might be below the level needed to allow maximum sustainable yield. Because it is often difficult to identify a species of fish after butchering, these organizations have documented a need for a portable, user-friendly field identification kit for billfish to assist in controlling illegal trafficking in these fish, as well as to accurately monitor catch statistics for these fish.

Methods used to identify butchered fishes have included isoelectric focusing of proteins (Lundstrom, 1980. J Assoc Off Anal Chem 53:7-9), restriction fragment length polymorphism analysis (Graves and McDowell, 1992, Ann Report Inter Comm Conserv Atl Tunas, SCRS/92IS0), polymerase chain reaction DNA sequence amplification and sequencing (Chow, 1992, Ann Report Inter Comm Conserv Atl Tunas, SCRS192166; Finnerty and Block, 1992, Molec Mar Biol Biotech 1:206-215), high performance liquid chromatography of sarcoplasmic proteins (Armstrong et al., 1992, Food Chemistry 44:147-155), and immunoassays using polyclonal antisera (Sutton et at., 1983, J Assoc Off Anal Chem 66:1164-1174). However, all of these test formats have the disadvantage of requiring substantial time and equipment in order to obtain results.

### SUMMARY OF THE INVENTION

The invention relates to the development of a quick and convenient immunoassay for identifying the presence of billfish tissue in a biological sample. The immunoassay is compatible with various fish types of fish samples including whole blood, serum, or tissue homogenates. In one aspect, this invention particularly relates to a lateral flow competitive immunoassay that utilizes a colloidal gold-monoclonal antibody probe for the qualitative detection of billfish serum albumin. The latter assay involves mixing a fish sample with a colloidal gold-monoclonal antibody conjugate and applying the mixture to one end of a plastic-backed nitrocellulose membrane strip with a filter paper slip at the opposite end as a wick. The presence of albumin in a target sample competes with adsorbed antigen to prevent the appearance of a pink color on the nitrocellulose membrane. A non-target sample yields a pink color when colloidal gold-labeled monoclonal antibodies bind to billfish albumin previously absorbed to the nitrocellulose. The assay requires only five minutes to perform and utilizes two inexpensive solutions.

In other aspects, the invention relates to a direct enzyme immunoassay of anti-billfish monoclonal antibodies following conjugation to horseradish peroxidase, a direct sandwich assay for sailfish identification using conjugated antibodies, and Nalge Nunc-Immno™ stick method for billfish identification.

Accordingly, the invention features a lateral flow immunoassay device for identifying the presence of tissue from a particular species of billfish in a test sample. This device includes a substrate (e.g., a plastic-backed nitrocellulose membrane) onto which a billfish specific antigen-containing sample has been immobilized. In one variation of the device, the substrate has a first end and a second end, the first end having thereon the immobilized billfish specific antigen-containing sample and the second end being adapted to receive a solution including an antibody that specifically binds the billfish specific antigen. In some cases, the solution can additionally include at least a portion of the test sample. As a negative control, a non-billfish specific antigen can be immobilized on the substrate. Various immunoassay devices of the invention have the solution applied on the substrate. In some cases, at least a portion of the antibody is specifically bound to the immobilized billfish specific antigen.

In another aspect, the invention features a kit for identifying the presence of tissue from a particular species of billfish in a test sample. The kit includes a lateral flow immunoassay device having: a substrate onto which a billfish-specific antigen-containing sample has been immobilized; and a solution comprising an antibody that specifically binds the billfish-specific antigen.

Also within the invention are methods for identifying the presence of tissue from a particular species of billfish in a test sample. On such method includes the steps of: (A) providing the test sample and a substrate onto which a billfish-specific antigen-containing sample has been immobilized; (B) preparing an antibody-test sample mixture by mixing the test sample with an antibody that specifically binds the billfish-specific antigen; and (C) applying the antibody-test sample mixture to the substrate. Another method includes the steps of: (A) providing the test sample and a substrate; (B) immobilizing at least a portion of the test sample on the substrate; (C) providing an antibody that specifically binds a billfish-specific antigen; and (D) applying the antibody to the substrate.

In the devices, kits, and methods of the invention, the billfish-specific antigen can be a billfish serum albumin such as a sailfish serum albumin, a blue marlin serum albumin, or a white marlin serum albumin. Antibodies used in the invention can be detectably labeled, for example, by conjugation to a gold particle such as one having a diameter of between 20-40 nm.

By the term “billfish-specific antibody” is meant an antibody that binds a particular billfish-specific antigen, and displays no substantial binding to other antigens except those that share the same antigenic determinants as the particular billfish-specific antigen. The term includes polyclonal and monoclonal antibodies.

As used herein, “bind,” “binds,” or “interacts with” means that one molecule recognizes and adheres to a particular second molecule in a sample, but does not substantially recognize or adhere to other structurally unrelated molecules in the sample. Generally, a first molecule that “specifically binds” a second molecule has a binding affinity greater than about 10⁵ to 10⁶ moles/liter for that second molecule.

The phrase “detectably labeled,” with regard to an antibody, is intended to encompass direct labeling of the antibody by coupling (i.e., physically linking) a detectable substance to the antibody. In some cases, the phrase also encompasses indirect labeling of the antibody by reactivity with another reagent that is directly labeled. Examples of indirect labeling include detection of a primary antibody using a fluorescently labeled secondary antibody and detection of a biotin-labeled primary antibody with fluorescently labeled streptavidin.

Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions will control. In addition, the particular embodiments discussed below are illustrative only and not intended to be limiting.

### DETAILED DESCRIPTION

The invention encompasses devices and methods relating to an immunoassay for detecting the presence of billfish in a test sample. The devices and methods of the invention utilize antibodies that specifically recognize a billfish-specific antigen such as a billfish serum albumin. The below described preferred embodiments illustrate adaptations of these devices and methods. Nonetheless, from the description of these embodiments, other aspects of the invention can be made and/or practiced based on the description provided below.

### Biological Methods

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies and immunoassays) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

### Immunoassay Devices

In brief overview, an immunoassay device for detecting the presence of billfish tissues in a sample includes a substrate onto which a billfish-specific antigen (e.g., a billfish serum albumin) is immobilized. The substrate can be any type of material to which a protein can be immobilized and which allows a fluid to flow through it. A particularly preferred substrate is a plastic-backed porous nitrocellulose membrane. The nitrocellulose allows immobilization of a protein. Its porosity allows fluid flow through the membrane, while the plastic backing prevents fluid from flowing through the backed side of the membrane. For ease of handling, the substrate may be partially enveloped with a rigid cover that may be made of plastic or another suitable material. The cover should have at least one aperture through which a sample can be applied to the substrate, and may optionally include a transparent or translucent window (e.g., composed of clear plastic), through which color development on the substrate can be observed.

Preferred devices of the invention are configured for either direct or competitive immunoassays. For direct immunoassays, a test sample is immobilized at one end of a substrate (usually rectangular and about 2 to 5 cm×5 to 15 cm). A solution of detectably labeled anti-billfish antibody is then applied to the substrate at the end of the substrate opposite of where the test sample was immobilized. By capillary action, the antibody solution migrates towards the immobilized test sample. This capillary action can be facilitated using a filter paper wick attached to the end of the substrate containing the immobilized sample. If the test sample contains a billfish antigen (e.g., billfish serum albumin) to which the anti-billfish antibody binds, the antibody will localize to the site of the immobilized protein through an antibody-antigen binding reaction. As the antibody has conjugated thereto a detectable label, visualization of the detectable label (e.g., observable as a red or pink line if a colloidal gold is used as the detectable label) on the substrate at the site of the immobilized test sample indicates that the test sample includes a billfish antigen. As a positive control, a sample known to include the billfish antigen can be immobilized on the substrate next to the immobilized test sample. Similarly, as a negative control, a sample known not to include the billfish antigen (e.g., a non-billfish serum albumin) can be immobilized on the substrate next to the immobilized test sample.

A competitive immunoassay device of the invention includes a substrate onto one end of which has been immobilized a billfish-specific antigen (e.g., a billfish serum albumin). The substrate is adapted for receiving a liquid sample including a solution of a test sample mixed with a detectably labeled anti-billfish antibody that specifically binds the billfish-specific antigen. Typically, the liquid sample is applied to the substrate at the end of the substrate opposite of where the billfish-specific antigen was immobilized. If the test sample contains a billfish antigen to which the anti-billfish antibody binds, much of the antibody in that sample will be bound to the antigen in the sample. This bound antibody will be unavailable for binding other billfish-specific antigens. After being applied to the substrate, the liquid sample will migrate towards the immobilized billfish-specific antigen. If the test sample does not include a billfish-specific antigen to which the antibody binds, the antibody will bind the immobilized billfish specific antigen on the substrate. Visualization of the detectable label (e.g., observable as a red or pink line if a colloidal gold is used as the detectable label) on the substrate thus indicates that the test sample does not include a billfish antigen. As indicated above, if the test sample does include a billfish-specific antigen to which the antibody binds, the antibody will be unavailable for binding the billfish specific antigen immobilized on the substrate. In this case, very little or no detectable label will be observed on the substrate.

As with the direct immunoassay devices, positive and negative controls can be employed in the competitive immunoassay devices. For example, as a positive control, in place of the test sample, a sample known to include the billfish antigen can be mixed with the antibody before application to the substrate. In the same manner, as a negative control, a sample known not to include the billfish antigen can be mixed with the antibody before application to the substrate. When run in parallel with the test sample, little or no detectable label should be observed on the positive control substrate, while the detectable label should be readily observable on the negative control substrate. As the absence of a signal indicates a positive result in the competitive immunoassay devices, inclusion of a positive and especially a negative control is preferred.

### Test Samples

The immunoassays of the invention are used for analyzing test samples with unknown billfish content. Test samples compatible for use with the devices of the invention include any liquid sample. Solid samples can be homogenized and mixed with a liquid (a buffered salt solution) to make them suitable for use with the devices. Typically, test samples to be analyzed according to the invention would be those obtained during field operations to assess whether butchered fish parts were derived from a billfish. As such, these samples would in most cases include materials from fishes, such as whole blood, serum, or solid tissue samples (e.g., filets).

### Billfish-Specific Antigens

The immunoassays of the invention are primarily designed to identify billfish species. Examples of billfish species include without limitation: striped marlin, Pacific blue marlin, Atlantic blue marlin, Pacific sailfish, Atlantic sailfish, spearfish, and white marlin. To determine whether a particular type of billfish tissue is in a test sample, the immunoassays of the invention use billfish-specific antigens and antibodies that specifically bind such antigens. The particular antigen used in the invention can be any that can be used to distinguish among fish types (e.g., species). Thus, antigens that are immunologically distinguishable among closely related species are preferred.

For example, a particularly preferred antigen for use in the invention is a billfish serum albumin. This antigen is advantageous for use in the present invention as a target antigen molecule for a number of reasons. First, serum albumin is the most abundant plasma protein, constituting about half of the total serum protein. Second, serum albumin is also a readily purified and highly immunogenic protein. Third, serum albumin is a rapidly evolving protein. This latter characteristic is especially important as the slight differences (e.g., in amino acid sequence) that exist among the serum albumin of different billfishes allow it to be used as a marker for differentiating among various species of billfishes. For example, epitopic differences between sailfish serum albumin and blue marlin serum albumin allow the two antigens to be distinguished by antibody-based methods.

As an example of billfish-specific antigen purification, blue marlin albumin was purified by heat ethanol fractionation. Five ml of blue marlin serum was diluted with an equal volume of distilled water. Ethanol (0.45 ml) and caprylic acid (66 mg of a 1:10 dilution) were added 1 drop-wise, and the pH of the solution was adjusted to 6.5 with 3N HCL. This solution was incubated at 68° C. in a water bath for 30 min and the pH was then adjusted to 4.4 with 3N HCL. The mixture was centrifuged at 10,000×g for 20 min and the supernatant fluid was removed, after which the pH was readjusted to 7.0 with 1N NaOH and the supernatant fluid was filter sterilized and frozen at −20° C.

### Antibodies

Billfish-specific antigens (or immunogenic fragments or analogs thereof) can be used to raise antibodies useful in the invention. In general, to make such antibodies, billfish-specific polypeptide antigens are mixed with an adjuvant, and injected into a host mammal. To boost immunogenicity, antigens can optionally be coupled to a carrier protein, such as KLH, as described in Ausubel et al., supra. Antibodies produced in that animal can then be purified by antigen affinity chromatography. In the assays described below, antigen affinity purification of the antibodies was an important step in promoting the development of a strong signal in the assay devices.

Various host animals can be immunized by injection with a billfish serum albumin or an antigenic fragment thereof. Commonly employed host animals include rabbits, mice, guinea pigs, and rats. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin, and dinitrophenol. Other potentially useful adjuvants include BCG (bacille Calmette-Guerin) and Corynebacterium parvum.

Polyclonal antibodies are heterogeneous populations of antibody molecules that are contained in the sera of the immunized animals. Antibodies for use within the invention therefore include polyclonal antibodies and, in addition, monoclonal antibodies, single chain antibodies, Fab fragments, F(ab′)₂ fragments, and molecules produced using a Fab expression library. Monoclonal antibodies, which are homogeneous populations of antibodies to a particular antigen, can be prepared using the billfish-specific antigens described above and standard hybridoma technology (see, for example, Kohler et al., Nature 256:495, 1975; Kohler et al., Eur. J. Immunol. 6:511, 1976; Kohler et al., Eur. J. Immunol. 6:292, 1976; Hammerling et al., “Monoclonal Antibodies and T Cell Hybridomas,” Elsevier, N.Y., 1981; Ausubel et al., supra). In particular, monoclonal antibodies can be obtained by any technique that provides for the production of antibody molecules by continuous cell lines in culture such as described in Kohler et al., Nature 256:495, 1975, and U.S. Pat. No. 4,376,110; the B-cell hybridoma technique (Kosbor et al., Immunology Today 4:72,1983; Cole et al., Proc. Natl. Acad. Sci. USA 80:2026, 1983), and the EBV-hybridoma technique (Cole et al., “Monoclonal Antibodies and Cancer Therapy,” Alan R. Liss, Inc., pp.77-96, 1983). Such antibodies can be of any immunoglobulin class including IgG, IgM, IgE, IgA, IgD and any subclass thereof. A hybridoma producing a monoclonal antibody of the invention may be cultivated in vitro or in vivo. The ability to produce high titers of monoclonal antibodies in vivo makes this a particularly useful method of production.

Once produced, polyclonal or monoclonal antibodies can be tested for specific billfish antigen recognition by Western blot or immunoprecipitation analysis by standard methods, for example, as described in Ausubel et al., supra. Antibodies that specifically recognize and bind to billfish-specific antigens are useful in the invention. To remove reactivity of a polyclonal anti-billfish antibody against an antigen of a particular species, the antibody can be adsorbed against the antigen of the particular species prior to use.

Preferably, species-specific antibodies of the invention are produced using fragments of a billfish antigen that lie outside highly conserved regions and appear likely to be antigenic, by criteria such as high frequency of charged residues. Cross-reactive anti-billfish antibodies are produced using a fragment of a billfish-specific antigen that is conserved amongst several different billfish species but not in other types of fish. Species-specific anti-billfish antibodies are preferred in assays for identifying the presence of a particular species of billfish in a test sample; whereas cross-reactive anti-billfish antibodies are preferred in assays for detecting the presence of any billfish species in a sample (as opposed to detecting only a particular species of billfish).

Techniques described for the production of single chain antibodies (U.S. Pat. Nos. 4,946,778, 4,946,778, and 4,704,692) can be adapted to produce single chain antibodies against billfish-specific antigen, or a fragment thereof. Single chain antibodies are formed by linking the heavy and light chain fragments of the Fv region via an amino acid bridge, resulting in a single chain polypeptide.

Antibody fragments that recognize and bind to specific epitopes can be generated by known techniques. For example, such fragments include but are not limited to F(ab′)₂ fragments that can be produced by pepsin digestion of the antibody molecule, and Fab fragments that can be generated by reducing the disulfide bridges of F(ab′)₂fragments. Alternatively, Fab expression libraries can be constructed (Huse et al., Science 246:1275, 1989) to allow rapid and easy identification of monoclonal Fab fragments with the desired specificity.

In order to make the results of the immunoassays observable, the antibodies used in the invention are conjugated to a detectable label. A variety of detectable labels are known including fluorescent labels, radioactive labels, enzymatic labels, and colorimetric labels. Based on experimental results, a particularly preferred detectable label is colloidal gold, especially colloidal gold particles having a diameter in the range of about 20 to 40 nm. Conjugation of gold particles to antibodies and the use of such labeled antibodies is described in U.S. Pat. No. 5,656,503. For some of the antibodies described below, the gold particle conjugation step was performed at an acidic pH as no or only poor coupling was observed when this step was performed at a neutral pH.

For use in the invention, anti-billfish antibody-producing hybridomas were made as follows. A Balb/c mouse was immunized intraperitoneally with Ribi adjuvant and 100 ug of billfish (e.g., Blue marlin) albumin dissolved in PBS, pH 7.1. Three to four weeks later, the animal was boosted with Ribi adjuvant and an additional 100 ug of blue marlin albumin. Test bleeds were performed 7-10 days later and titered by ELISA to an endpoint dilution of 1:10,000. A final intravenous boost was performed three days before spleen harvest and at least three weeks after the previous injection.

The mouse myeloma X-63 was thawed from liquid nitrogen and cultured with H-Y medium and 10% fetal calf serum (FCS) (Sigma-Aldrich, St. Louis, Mo.). The X-63 cells were checked for resistance to 8-azaguanine and sensitivity to HAT medium. Before the fusion, X-63 cells were counted. Adherent cells were suspended in 50 ml of HY medium. The volume was adjusted to contain X-63 cells amounting to approximately half the number of spleen cells.

The immunized mouse was sacrificed and its spleen was removed aseptically and placed in a 100 mm tissue culture dish containing 10 ml of H-Y medium w/o serum, prewarmed to 37° C. The spleen was teased apart using two 21-gauge needles on 5 ml syringes until most of the cells had been released. Cell clumps were disrupted by repeatedly pipetting the medium. The cells and medium were transferred into a sterile centrifuge tube, leaving behind the larger pieces of tissue. Larger cell clumps were allowed to settle to the bottom of the tube over a two-minute period. The supernatant was carefully removed from the sediment and transferred to a fresh centrifuge tube. The cell suspension was centrifuged at 400×g for 10 min and the supernatant was discarded. One ml of red blood cell lysing solution, diluted with 15 ml of H-Y medium, was added and the pellet was gently mixed for 1 min. The cell suspension was centrifuged at 400×g for 10 min and the supernatant was decanted. The pellet was resuspended in 10 ml of H-Y medium and a cell count was performed. A spleen from an immunized mouse contained approximately 5×10⁷ to 2×10⁸ lymphocytes.

Both sets of X-63 and spleen cells were centrifuged at 400×g for 10 min and the supernatant were vacuumed off. Spleen cells were resuspended in H-Y medium and combined with X-63 cells. The combined volume was brought to 50 ml. The combined cells were spun out at 400×g for 10 min, the supernatant was carefully vacuumed off while trying to remove the entire medium. The pellet was dispersed by gently tapping the tube in a beaker containing 37-40° C. water. One ml of 37° C. prewarmed polyethylene glycol PEG (fusion-tested, sterile PEG 1500; two g in 75 mM Hepes [Boehringer Mannheim GmbH, W. Germany]) was added slowly over a one min period. The cell mixture was stirred gently with the pipette tip to mix the PEG and cells. The bottom of the tube was held in the water bath and shook gently for exactly one min. One ml of H-Y medium without serum was added slowly over one min and then 20 ml of the same medium was added slowly over five min; this mixture was centrifuged at 400×g for 15 min. The supernatant fluid was discarded and the cell pellet was resuspended gently and slowly in 50 ml of HY medium with 20% FCS and plated at 100 ml/well in sterile microwell plates (Becton Dickinson, Franklin Lakes, N.J.). Cells were switched to complete HAT medium with 20% FCS after 24 hr at 37° C. After two days, hybridomas were spun down and resuspended with fresh H-Y medium with 10% FCS. After one week, the hybridomas were counted and 10⁶ cells were resuspended in 0.5 ml PBS, pH 7.1.

To make a polycloanl antibody preparation, these hybridomas were injected into pristane primed (10-60 days before use) Balb/c mice intraperitoneally. Ascites fluid was collected and purified for polyclonal antibody by thiophilic adsorption chromatography. The ascites was pretreated gently by dissolving 87 mg of potassium sulfate per ml of sample. Care was used to avoid foaming in order to prevent denaturation of the immunoglobulins. The sample was centrifuged at 10,000×g for 20 minutes and the clean supernatant fluid was removed carefully. The storage buffer on a T-Gel™ (Pierce Chemical Co., Rockford, Ill.) adsorbent column was allowed to drain, and the column equilibrated with 12 ml of T-Gel™ binding buffer. The pretreated ascites fluid that contained the antibody of interest was allowed to completely enter the gel. The column was washed with up to 38 ml of T-Gel™ binding buffer and the column effluent was collected as three ml aliquots of not-bound (NB) fractions. The column was eluted with 36 ml of T-Gel™ elution buffer and the column effluent was collected as three ml bound (B) fractions. The adsorbent column was regenerated by adding 15 ml of eight molar guanidine hydrochloride solution, and the column was allowed to drain. The column was then rinsed with 30 ml of degassed, deionized water followed by six ml of 1× storage buffer, and the column was allowed to drain. The lower cap was installed on the column, three ml of 1× storage buffer were added and the top cap to the column applied. The column was stored refrigerated at 4° C. in an upright position. Ascites fluids containing monoclonal antibodies and polyclonal antibodies were purified by this method. The absorbance of eluded proteins was read at 280 nm. All bound fractions with an optical density greater than 0.100 were pooled and concentrated by centrifugal dialysis at 3,500×g using an ultrafree-CL centrifugal filter (Sigma Chemical Co., St. Louis, Mo.) with a molecular weight cut-off of 30 KD. The purified antibodies were stored at 4° C.

For use in the invention, monoclonal antibodies were produced from established hybridomas as follows. Cells from three different hybridomas producing monoclonal antibodies (E8, WMB9, and WML4D7, respectively) which reacted with billfish serum albumin, were grown to confluence. They were adjusted to concentrations of 1.14×10⁶, 2.88×10⁶ and 1.73×10⁶ cells per ml, respectively, washed in H-Y medium, resuspended in 0.5 ml of phosphate buffered saline (PBS), and then injected intraperitoneally into pristane-primed mice. For each hybridoma, four 8-10 week old Balb/c mice were primed with 0.5 ml pristane (Sigma Chemical Company, St. Louis, Mo.) by intraperitoneal injection three to four weeks prior to the injection of the hybridomas. Two to four weeks after injecting the hybridomas, when the animals were the size of sexually mature females, ascites fluid was aspirated. The ascites fluid was incubated at 37° C. for 30 min, centrifuged at 2000×g for 15 min, and the supernatant fluid was then removed. Four parts of the latter were mixed with one part of Cleanascites™ (Affinity Technology, Inc. New Brunswick, N.J.). After incubation at room temperature for 10 minutes with occasionally shaking, the material was centrifuged again at 2000×g for 15 min. The supernatant fluid was dispensed into cryo-tubes and frozen at −75° C.

### Lateral Flow and Other Assays

The invention provides several varieties of immunoassay devices including lateral flow devices as well as ELISA (enzyme-linked immounosorbent assay)-based devices and ImmunostickT™-based devices. As described above, lateral-flow direct and competitive immunoassay devices are particularly preferred for their ease of use and rapidity of results. See, e.g., U.S. Pat. Nos. 5,075,078; 5,096,837; 5,229,073; 5,354,692; 5,656,503; and 6,316,205. Many currently marketed lateral flow assays use colloidal gold conjugates as a detector reagent. In typical colloidal gold lateral flow assay devices, colloidal gold conjugates are placed at one end of a membrane strip. Upon coming into contact with a biological sample (e.g., urine, whole blood, serum, plasma, the extraction reagent or the running buffer), the colloidal gold conjugate quickly becomes soluble and binds to antigen or antibody in the sample and moves across the membrane by capillary migration until it reaches the test zone where color development can be observed by the naked eye. The procedures usually can be completed within 2-3 minutes.

In competitive assays, the immobilized antigen at the test zone competes with the antigen that may be present in sample for limited antibody binding sites on the antibody-colloid gold conjugate. In the absence of antigen in the sample, the colored antibody-colloid gold conjugate moves along with the sample by capillary action along the membrane until it reaches the immobilized antigen at test zone and forms a visible line, which shows a negative detection for the tested antigen. When the tested antigen is present in the sample, the antigen competes with the immobilized antigen at the test zone for the limited antibody sites on the antibody-colloid gold conjugate. If an adequate amount of antigen is present, it will fill the limited antibody binding sites and prevent the binding of the colored antibody-colloid gold conjugate to the immobilized antigen at the test zone. In this case, absence of the color band at test zone indicates a positive result.

A preferred lateral flow device of the invention is prepared as follows. Purified sailfish serum albumin is dissolved in TBS (tris-buffered saline), pH 7.4, at a concentration of 20 ug/ml. Five ul of this solution is immobilized onto a plastic-backed nitrocellulose membrane, strip type FF85, 25 mm width, with a pore size of 0.45 um (Schleicher and Schuller, Keene, NH). The membrane is incubated at 37° C. for 15 minutes or at room temperature for 30 minutes in order to dry it. Non-specific binding sites on the membrane are blocked by the addition of 1% cold water fish gelatin/TTBS (TBS with Tween 20 added to 0.05%), pH 7.4, for 30 minutes followed by washing 2× with TTBS for 3 minutes.

Target and non-target fish samples are centrifuged at 5,000×g for 5 minutes. If the assaying sample is tissue, it can be homogenized in TBS, pH 7.4, at a weight:volume ratio of one:five using a ground glass homogenizer. Twenty to 50 ul of the clarified sample is mixed with 10 to 15 ul of the colloidal gold/antibody conjugate with an O.D.₅₃₇ of 10.0 and is allowed to incubate for 5 minutes before being applied to one end of the nitrocellulose strips. A visible result is apparent after two to three minutes. Since this is a competitive assay, the lack of color development, as compared to the positive control, indicates a positive identification.

As described further in the Examples section, the invention also provides other assays for identifying the presence of billfish in a test sample. These include a direct enzyme immunoassay of anti-billfish monoclonal antibodies following conjugation to horseradish peroxidase, a direct sandwich assay for billfish identification using conjugated antibodies, and Nalge Nunc-Immuno stick™-based method for billfish identification. Methods and material for use in such assays are described below.

### EXAMPLES

The present invention is further illustrated by the following specific examples. The examples are provided for illustration only and are not to be construed as limiting the scope or content of the invention in any way.

### Example 1

### A Lateral Flow Direct Immunoassay

Five hundred ng each of sailfish albumin, blue marlin albumin and bovine serum albumin was dispensed in lines on a nitrocellulose strip with a plastic backing. The strip was dried, blocked, washed and dried successively. 20 ul of colloidal gold-labeled sailfish specific monoclonal antibody was applied at one end of the strip. A filter paper strip was utilized as a wick at the other end of the nitrocellulose strip. A few minutes after applying the antibody solution, a red color developed at the line where the sailfish albumin was applied indicating positive detection. In comparison, no color development was noted at the lines where the blue marlin albumin and bovine serum albumin was applied. Therefore, the gold probe reacted on a sailfish species-specific basis.

### Example 2

### Sensitivity and Specificity Tests Carried out for Purified Fish Serum Albumin by a One-Step Lateral Flow Competitive Immunoassay

Purified Sailfish albumin was used to evaluate the sensitivity of a lateral flow competitive immunoassay. One hundred ng of purified sailfish albumin was immobilized onto plastic-backed nitrocellulose strips. Different amounts of purified sailfish albumin (0, 20, 50, 100, 1000 ng) were mixed with a colloidal gold-labeled sailfish-specific monoclonal antibody solution. This antibody-albumin mixture was incubated for 5 min before being applied to one end of the nitrocellulose strips to allow antigen-antibody binding to proceed. Four hundred ng of purified blue marlin serum albumin was tested as a negative control. After a few minutes, a pink-colored line appeared in both the blue marlin serum albumin negative control sample and the 0 ng sailfish albumin sample. The appearance of this strong signal indicates a negative result (i.e., no Sailfish was present in the test sample). In comparison, in the 20, 50, 100, 1000 ng sailfish albumin samples, little or no pink color was detected. This signal (i.e., little or no color development) indicates a positive result, i.e., that sailfish was present in the test sample. The data from this experiment also showed that 100 ng of purified sailfish albumin could compete with 100 ng of immobilized antigen for 10 ul of colloidal gold-monoclonal antibody conjugate.

### Example 3

### Specificity Tests Carried out for Fish Heart Tissue Samples by a Lateral Flow Competitive Immunoassay

Fifteen billfish heart tissue samples, including 3 each of striped marlin (sample No. 13, collected in Australia, 1990), Pacific blue marlin (sample No. 4-6, collected in Kona, Hi., 1992), Atlantic blue marlin (sample No. 7-9, collected in Jamaica, 1992), Pacific sailfish (sample No. 10-12, collected in Cabo San Lucas, Mexico, 1991), and Atlantic sailfish (sample No. 13-15, collected in Islamorada, Fla., 1991), were used to evaluate the specificity of a lateral flow competitive immunoassay. A 1:6 (w/v) tissue homogenate of these samples was prepared by homogenizing 0.5 g of each heart tissue sample with a glass pestle in 3 ml TBS pH 7.4 supplemented with 100 μg/ml merthiolate. Twenty μl of each sample was mixed with 10 μl colloidal gold sailfish-specific monoclonal antibody conjugate (O.D.₅₃₇ of 10.0 for a 40 nm probe) in the lateral flow competitive test.

It was shown that all the nine samples from striped marlin, Pacific blue marlin and Atlantic blue marlin yielded a distinct red/purple precipitating line at the test zone, denoting the negative detection (i.e., indicating that no Sailfish was present in the samples). In comparison, three samples from Atlantic sailfish showed positive results with an absence of the precipitating red/purple line at the test zone; and three samples from Pacific sailfish developed very weak signals at the test zone (interpreted also as positive results).

### Example 4

### Specificity Tests Carried out for Fish White Muscle Tissue Samples by Lateral Flow Competitive Immunoassay

Five billfish white muscle samples, including three sailfish samples (sample SF, SF92, and SF0413), one blue marlin sample, and one white marlin sample were used to evaluate the specificity of a lateral flow competitive immunoassay. The SF, SF92, blue marlin and white marlin samples were stored in a walk-in freezer for years (e.g., sample SF92 was collected in 1992) prior to testing; while sample SF 0413 was freshly prepared and tested within 24 hours. A 1:5 (w/v) tissue homogenate of each of these samples, except SF0413, was prepared by homogenizing 0.5 g of each heart tissue with a glass pestle in 3 ml TBS pH 7.4 supplemented with 100 ug/ml merthiolate. The SF0413 sample homogenate was prepared by homogenizing 1 g of tissue in 10 ml (1:10 wlv) TBS as above. Fifty μl of each sample (40 ul for sample SF0413) was mixed with 15 μl of colloidal gold sailfish albumin-specific monoclonal antibody conjugate (O.D.₅₃₇ of 10.0 for a 20 nm probe) in the lateral flow competitive test.

It was shown that samples from white marlin and blue marlin yielded distinct precipitating line at the test zone denoting a negative detection. Three samples from Atlantic sailfish showed a positive result with an absence of the precipitating red/purple line at the test zone.

### Example 5

### Direct Enzyme Immunoassay of Anti-Sailfish Monoclonal Antibodies Following Conjugation to Horseradish Peroxidase

Sailfish serum albumin diluted 1:1000 in TBS, pH 7.9, and sailfish heart tissue homogenates diluted 1:400 in TBS, were adsorbed to separate ELISA plates. Aliquots (100 ul per well) were incubated for one hour at room temperature. The wells were rinsed three times with TBS. All wells were completely filled with 0.5% gelatin in TTBS containing 0.5% tween 20 (blocking solution) and incubated for one hour at room temperature. The wells were rinsed three times with TTBS, and 100 ul of purified monoclonal or polyclonal antibodies, serially diluted in antibody buffer (0.15% gelatin in TTBS), were added. The initial dilution of antibodies was 1:10 with continued serial dilutions of 1:4, to obtain a final dilution of 1:163,840. After a one hour incubation at room temperature and three washings with TTBS, 100 ul of secondary antibody horseradish peroxidase conjugated goat anti-mouse (GAM-HRP, Sigma Chemical Company, St. Louis, Mo.) solution were added to each of the wells and incubated for one hour at room temperature. The plate was washed five times with TTBS and 100 ml of 3,3′,5,5′-tetramethylbenzidine (TMB) solution (Southern Biotechnology Associates, Inc. Birmingham, Ala.) was added. The absorbance was measured at 405 nm by using a microplate reader. Two positive controls were established, one by binding goat antimouse/horseradish peroxidase (GAM-HRP) directly to the wells and the other by first adding the monoclonal antibody M2D1 (See, et al., Hybridoma 11:333-338, 1992), followed by GAM-HRP. The negative control consisted of blocking the wells without the antigen.

### Example 6

### Direct Sandwich Assay for Sailfish Identification Using Conjugated Antibodies

Purified monoclonal antibody M2D1 at a concentration of 1.2 mg/ml was diluted to 1:1000 using TBS. One hundred ul of diluted M2D1 was adsorbed to an ELISA plate for one hour at room temperature to act as the species-specific capture antibody. All wells were coated with blocking solution for one hour and then washed three times with TTBS. Purified sailfish serum (1:1000) was added to each well as a source of albumin. After incubation for one hour at room temperature, the wells were rinsed three times with TTBS. Serially diluted HRP conjugated monoclonal or polyclonal antibodies, with the dilution starting at 1:100 through 1:12,800, were added to the wells. After another one-hour incubation period at room temperature, the plate was washed three times with TTBS. TMB substrate was added to allow a visible color change. The absorbance was read at 405 nm after the reaction was stopped with 1N HCL. One positive control (GAM-HRP coated well) and one negative control (a well treated with blocking solution) were run on each plate.

### Example 7

### Determination of the Epitope Specificity of Monoclonal Antibodies

An ELISA plate was coated with 100 ul of diluted (1:4000) sailfish serum for one hour, followed by washing three times with TTBS and then blocking. After washing the plate three more times with TTBS, it was incubated with serial dilutions of unlabeled, competing monoclonal antibody for one hour at room temperature. The excess antibody was removed by washing three times with TTBS. The plate was incubated with diluted (1:800) HRP-labeled test monoclonal antibody for one hour. Controls consisting of unlabeled test monoclonal antibody were used to demonstrate maximal competition and labeled test monoclonal antibody without any competitor to demonstrate zero competition were included. The procedure was completed as described above, using TMB as the substrate.

### Example 8

### Cross-Reactivity Test with Two Antibody Conjugates Added Simultaneously

One hundred ul of diluted (1:4000) sailfish serum was adsorbed to an ELISA plate for one hour at room temperature. All wells were coated with blocking solution for one hour and washed three times with TTBS. Two diluted (1:800), HRP-labeled monoclonal antibodies were pooled together and added to each well. The procedure was completed as described above. Controls consisting of single monoclonal antibody conjugates were run in parallel.

### Example 9

### Evaluation of the Amount of Enzyme-Conjugated to Antibody on the Signal Strength of the Immunoassay

The relative level of HRP incorporation can be controlled by varying parameters such as the molar excess of peroxidase, buffer, and pH used in the conjugation. Different molar ratios (4:1, 2:1, 1:1, 0.5:1) of peroxidase coupled to an anti-billfish monoclonal antibody WML4D7 were tested. Where sailfish serum was the antigen used in a direct plate immunoassay and its presence detected with dilutions (1:500 to 1:16000) of the four different HRP conjugates. The optimal molar ratio of peroxidase to antibody was 4:1. The antibody conjugate prepared at this ratio yielded the strongest signal. Two additional anti-billfish monoclonal antibodies (E8 and WMB9) were conjugated at this optimal ratio. Usually lower ratios are required for some antibodies so that their active binding sites are not covered with enzyme.

### Example 10

### Evaluation of the Activity of Monoclonal Antibody Conjugates (E8-HRP, WMB9-HRP, WML4D7-HRP) in Various Assay Formats

Monoclonal antibodies E8, WMB9, WML4D7, which were produced against billfish albumin, retained their activity after purification and conjugation. In both the direct and indirect sandwich assays, anti-sailfish specific monoclonal antibody M2D1 was coated to an ELISA plate as capture antibody. The mouse anti-billfish polyclonal antibody and goat antimouse HRP were used to generate the signal in the indirect sandwich assay at various dilutions. The anti-billfish monoclonal antibody conjugates were compared at these same dilutions. E8-HRP, WMB9-HRP, WML4D7-HRP, when employed in the direct assay, provided signal strength nearly equivalent to the indirect assay.

### Example 11

### Determination of Epitope Overlap of Three Anti-Billfish Monoclonal Antibodies

An important characterization test of any panel of antibodies was the analysis of whether they react to the same, close or totally different epitopes. The simplest way was to label one of the antibodies directly and to allow it to compete for antigen with unlabeled antibodies of other sources (Liddell, J. E. and A. Cryer. 1991. A Practical Guide to Monoclonal Antibodies. John Wiley & Sons Ltd, West Sussex, England.). In a competitive ELISA, unlabeled monoclonal antibody E8 was applied as a competitor before enzyme labeled monoclonal antibody WMB9 was allowed to bind. The signal was equivalent to the exclusive WMB9 conjugate binding without E8 competition. Thus it was deduced that E8 and WMB9 were directed towards different epitopes. Meanwhile enhanced signal strength gained by using E8-HRP and WMB9-HRP together also supported this conclusion. It was the same case with another combination of E8 and WML4D7. As to the combination of WMB9 and WML4D7, the binding of HRP labeled antibody (WMB9 or WML4D7) was partially inhibited by the other unlabeled antibody (WML4D7 or WMB9). This result reflected the existence of competition between the antibodies for the same or very close epitopes. Alternatively, the binding of one antibody caused steric alterations in the antigen molecule, which prevented the other antibody from binding to its own distinct epitope. When WMB9-HRP and WML4D7-HRP were pooled together, a weaker signal was observed. These two antibodies may thus react with the same epitope.

### Example 12

### The Stability of Monoclonal Antibody Conjugates

Following initial conjugation with EZ-link™ plus activated peroxidase, three anti-billfish monoclonal antibodies (E8, WMB9, WML4D7) were tested for activity and then stored for five months at −20° C. The conjugates were re-tested for their activity against sailfish versus non-target fish samples by utilizing a direct sandwich assay. These three antibody conjugates retained nearly the same activity. This result demonstrated that the storage in conjugation buffer with a final concentration of 50% glycerol at −20° C. was suitable for long term storage and the antibody conjugates were stable.

### Example 13

### Nunc-Immuno™ Stick Methods

The Nunc-Immuno™ stick was developed for ELISA tests in the field, away from the laboratory environment. The test system consists of a tube and stick with a paddle which feature in giving reliable semiquantitative results with positive and negative controls at the same time on one immunostick. The immunostick provides two choices of MaxiSorp™ or PolySorp™ surface of the stick. The polysorp surface is excellent for binding of various antigens while the maxisorp surface has substantial affinity to antibodies and proteins. In a particular application, the maxisorp surface was chosen for evaluation (as sailfish specific monoclonal antibody M2D1 was coated onto the immunostick paddle surface as capture antibody). In the immunostick-based direct sandwich assay, all of the three monoclonal antibody conjugates (E8-HRP, WMB9-HRP, WML4D7-HRP) displayed a visible precipitate blue color towards sailfish serum samples while no visible color change was observed towards non-sailfish (Blue marlin and tuna) samples.

The anti-billfish polyclonal antibody conjugate was also evaluated by this method. Due to the fact that Blue marlin serum was used as antigen source during the preparation of polyclonal antibody, the purified polyclonal consisted of certain percentage of antibody population reactive to Blue marlin serum albumin but yet to Sailfish serum albumin. This explained that polyclonal conjugate binding resulted in a weaker signal compared to WMB9-HRP and WML4D7-HRP labeling.

Different incubation times 5min, 8min, 10 min, respectively, were tested in this immunostick based direct sandwich assay for antigen capture and monoclonal antibody conjugate binding. Although a 10 min incubation at room temperature yielded stronger color development than 8 min and 5 min incubations, 5 min incubation time for both steps was enough to generate a obvious color change to target species. Thus the whole identification procedure could be completed within 15 min.

A simple method for the attachment of most reactants possessing free amino groups was the pretreatment of polysyrene with glutaraldehyde (Barrett, U.S. Pat. No. 4,001,583). Polystyrene was activated at a relatively low pH where glutaraldehyde had no strong tendency to react or to form polymers. After the addition of the immunoreactant, the pH was raised to 8 to increase the reactivity of glutaraldehyde. Pretreatment of antibodies with glutaraldehyde prior to coating could also decrease their desorption and gave a higher detectability in enzyme-based immunoassays. The immunosticks pretreated with glutaraldehyde showed enhanced color development while other factors remained as constants.

In the immunostick based direct sandwich assay, when two monoclonal antibody conjugates (E8-HRP and WMB9-HRP, E8-HRP and WML4D7-HRP) which reacted with different epitopes were pooled together, an additive signal strength was achieved. This approach may be advantageously used in applications where the signal of a single monoclonal antibody conjugate is weak and also to shorten incubation time in a rapid identification procedure.

### Example 14

### Specificity of Immunostick-Based ELISA for Sailfish Identification

The effects of Immunosticks exposed to different billfish heart tissue homogenate samples were examined. Fifteen heart tissue samples of each billfish species (3 each for striped marlin, Pacific blue marlin, Atlantic blue marlin, Pacific sailfish, Atlantic sailfish, respectively) each from a different individual and four white muscle samples (Atlantic and Pacific sailfish) were tested. All Atlantic and Pacific sailfish tissue extracts and positive control developed visible blue color while non-target species extracts and negative control yielded no visually distinguishable color change. The optical density at 405 nm was easily obtained by eluting the blue color precipitation on the surface of immunostick with hydrochloric acid.

### Other Embodiments

While the above specification contains many specifics, these should not be construed as limitations on the scope of the invention, but rather as examples of preferred embodiments thereof. Many other variations are possible. For example, although much of the foregoing describes the methods for identifying billfish tissue in an unknown sample, other fish tissue such as tuna could be identified and distinguished from other species using antibodies are other reagents that specifically detect the other fish tissue using the devices and methods taught herein. Accordingly, the scope of the invention should be determined not by the embodiments illustrated, but by the appended claims and their legal equivalents.

What is claimed is:

1. A lateral flow immunoassay device for identifying the presence of tissue from a particular species of billfish in a test sample, the device comprising a substrate onto which a billfish specific antigen-containing sample has been immobilized.

2. The immunoassay device of claim 1, wherein the substrate comprises a nitrocellulose membrane.

3. The immunoassay device of claim 2, wherein the substrate comprises a plastic-backed nitrocellulose membrane.

4. The immunoassay device of claim 1, wherein the substrate has a first end and a second end, the first end having thereon the immobilized billfish-specific antigen-containing sample, and the second end being adapted to receive a solution comprising an antibody that specifically binds the billfish-specific antigen.

5. The immunoassay device of claim 4, wherein the solution further comprises at least a portion of the test sample.

6. The immunoassay device of claim 1, wherein the billfish-specific antigen is a billfish serum albumin.

7. The immunoassay device of claim 6, wherein the billfish serum albumin comprisess sailfish serum albumin.

8. The immunoassay device of claim 6, wherein the billfish serum albumin comprises blue marlin serum albumin.

9. The immunoassay device of claim 6, wherein the billfish serum albumin comprises white marlin serum albumin.

10. The immunoassay device of claim 4, wherein the solution is applied on the substrate.

11. The immunoassay device of claim 10, wherein at least a portion of the antibody is specifically bound to the immobilized billfish specific antigen.

12. The immunoassay device of claim 10, wherein the antibody is detectably labeled.

13. The immunoassay device of claim 12, wherein the detectably labeled antibody is conjugated to a gold particle.

14. The immunoassay device of claim 12, wherein the gold particle has a diameter of between 20-40 nm.

15. The immunoassay device of claim 1, wherein a non-billfish specific antigen has been immobilized on the substrate.

16. A kit for identifying the presence of tissue from a particular species of billfish in a test sample, the kit comprising:
a lateral flow immunoassay device comprising a substrate onto which a billfish-specific antigen-containing sample has been immobilized; and
a solution comprising an antibody that specifically binds the billfish-specific antigen.

17. The kit of claim 16, wherein the billfish specific antigen is a billfish serum albumin.

18. The kit of claim 17, wherein the billfish serum albumin is selected from the group consisting of sailfish serum albumin; blue marlin serum albumin; and white marlin serum albumin.

19. The kit of claim 16, wherein the antibody is detectably labeled.

20. The kit of claim 19, wherein the detectably labeled antibody is conjugated to a gold particle.

21. The kit of claim 20, wherein the gold particle has a diameter of between 20-40 nm.

22. The kit of claim 16, wherein a non-billfish specific antigen has been immobilized on the substrate.

23. A method for identifying the presence of tissue from a particular species of billfish in a test sample, the method comprising the steps of:
(A) providing the test sample and a substrate onto which a billfish-specific antigen-containing sample has been immobilized;
(B) preparing an antibody-test sample mixture by mixing the test sample with an antibody that specifically binds the billfish specific antigen; and
(C) applying the antibody-test sample mixture to the substrate.

24. The method of claim 23, wherein the billfish-specific antigen is a billfish serum albumin.

25. The method of claim 24, wherein the billfish serum albumin is selected from the group consisting of sailfish serum albumin; blue marlin serum albumin; and white marlin serum albumin.

26. The method of claim 23, wherein the antibody is detectably labeled.

27. The method of claim 26, wherein the detectably labeled antibody is conjugated to a gold particle.

28. The method of claim 23, wherein a non-billfish specific antigen has been immobilized on the substrate.

29. A method for identifying the presence of tissue from a particular species of billfish in a test sample, the method comprising the steps of:
(A) providing the test sample and a substrate;
(B) immobilizing at least a portion of the test sample on the substrate;
(C) providing an antibody that specifically binds a billfish-specific antigen; and
(D) applying the antibody to the substrate.

30. The method of claim 29, wherein the billfish-specific antigen is a billfish serum albumin.

31. The method of claim 30, wherein the billfish serum albumin is selected from the group consisting of sailfish serum albumin; blue marlin serum albumin; and white marlin serum albumin.

32. The method of claim 29, wherein the antibody is detectably labeled.

33. The method of claim 32, wherein the detectably labeled antibody is conjugated to a gold particle.

34. The method of claim 29, wherein a non-billfish specific antigen has been immobilized on the substrate.
